(19) United States
(12) Patent Application Publication
(10) Pub. No.: US 2002/0169563 A1
(43) Pub. Date: Nov. 14, 2002

(54) Linear and non-linear genetic algorithms for solving problems such as optimization, function finding, planning and logic synthesis

(76) Inventor: Maria Candida de Carvalho Ferreira, Acores (PT)

Correspondence Address: SUGHRUE, MION, ZINN, MACPEAK & SEAS, PLLC, 2100 Pennsylvania Avenue, N.W., Washington, DC, 20037-3213, US

(21) Appl. No.: 09/899,282

(22) Filed: Jul. 6, 2001

(30) Foreign Application Priority Data
Aug. 10, 2000 (PT) PT 102508

(51) Int. Cl. G06F 19/00; G01N 33/48; G01N 33/50

(52) U.S. Cl. 702/20

(57) ABSTRACT

The present invention is a mixed (linear and non-linear) genetic algorithm capable of learning and inventing. An initial population of linear chromosomes (linear entities) composed of genes containing the functions and arguments to a problem, is created and expressed as non-linear entities called expression trees. The non-linear entities are then executed, producing results. Then the results are assigned values and the respective individuals (linear entities and respective non-linear entities) are selected to reproduce according to these values. During reproduction, the linear entity or chromosome is subjected to one or several operators, namely, mutation, one-point recombination, two-point recombination, transposition, insertion and gene transposition. This way, new individuals are created which are in their turn executed, initializing a new cycle which is repeated as many times as necessary to discover a solution to the problem.

### PRIOR ART

This invention is related to the genetic algorithms and genetic programming (initially called non-linear genetic algorithms) and can be viewed as a synthesis of both systems with emergent properties.

In the history of life existed RNA entities capable of replication and some rudimentary enzymatic activity and, in fact, RNA can function both as genome and catalyst. Although possible, an RNA based life was condemned to very simple forms of life.

It is known that DNA is incapable of catalytic activity but is the ideal molecule to both store and transmit the genetic information provided the existence of enzymes capable of catalyzing the necessary reactions. The genetic information is then expressed as proteins which are capable of enzymatic activity.

Put very simply, in nature there is a division of labor between DNA and proteins: DNA is the storehouse of genetic information and the proteins are the expression of that information in the form of enzymes, structural proteins, antibodies, etc.

Genetic programming invented by J. Koza is analogous to an RNA World or Protein World, extremely complex and cumbersome to solve relatively simple tasks, whereas the genetic algorithms invented by J. Holland are analogous to a hypothetical DNA World: not so structurally complex but then incapable of solving a number of problems. The disadvantages of a system like genetic algorithms were pointed by many (see the works of J. Koza for a synopsis). Specifically, the simple language of chromosomes (usually 0's and 1's) and their fixed length make it difficult to apply this technique to more sophisticated problems.

With the invention of genetic programming, J. Koza solved partially these drawbacks by creating non-linear entities with different sizes and shapes allowing the application of evolutionary computation to new problems.

However, both genetic algorithms and genetic programming share a common problem: the created and manipulated entities function at the same time as genotype and phenotype, which not only limits considerably the performance of both techniques but also limits their application to relatively simple problems. As I said earlier, in the history of life on Earth, the RNA World turned out to be nonviable due to the great complexity necessary to solve extremely simple tasks; on the other hand, it is unlikely that a DNA World ever existed as this molecule is structurally very simple, thus incapable of catalytic activity. Although more flexible, both structurally and functionally, genetic programming is highly inefficient in terms of computational resources because genetic information is kept in a very complex structure, making the manipulation of this information extremely expensive. Genetic programming is similar to what would have happened if to reproduce ourselves we would have needed to make a copy of all the cells and constituents of our body instead of passing on uniquely our genome during reproduction. Thus, it is common for genetic programming to use huge populations to solve relatively simple problems, which greatly prevents its application to more complex problems.

In the present invention, the individuals are complex entities with emergent properties, such that the information necessary to the development of an individual is encoded as a simple linear message—the genome of the individual. As in nature, this genome is afterwards expressed as a complex entity with emergent properties, i.e. more complex both structurally and functionally than the chromosome in which it is encoded.

Thus, in the present invention there are two types of entities with different structures and functions: a genome or linear chromosome that is used to keep and transmit the genetic information to future generations, and a body called expression tree that is the expression of the genetic information encoded in the genome. This way, and similarly to nature, the present invention allows the creation of complex individuals of different sizes, shapes and properties despite their being encoded as linear chromosomes of fixed length. Thus, the manipulation of the genetic information, fundamental for evolution to occur and therefore fundamental for solving problems, is done as easily and simply as is done for the chromosomes of genetic algorithms. The modifications that took place during the creation of new descendants are tested whenever the genome of the individual is expressed and, as in nature, if the modification brings advantages to the descendent, the likelihood of surviving increases and therefore it has more chances of leaving offspring; the opposite happens if the modification decreases the individual's performance: this individual will leave less descendants or will be excluded from the population.

### CITED REFERENCES

U.S. Patent Documents:

U.S. Pat. No. 4,697,242. Adaptive Computing System Capable of Learning and Discovery. Sep. 29, 1987. Holland, J. H., and Burks, A. W.

U.S. Pat. No. 4,935,877. Non-Linear Genetic Algorithms for Solving Problems. Jun. 19, 1990. Koza, J. R.

Other Documents:

Holland, J. H. (1975). Adaptation in Natural and Artificial Systems. An Introductory Analysis with Applications to Biology, Control, and Artificial Intelligence. Ann Arbor, Mich.: University of Michigan Press.

Koza, J. R. (1992). Genetic Programming: On the Programming of Computers by Means of Natural Selection. Cambridge, Mass.: MIT Press.

O'Reilly, U-M, and F. Oppacher (1996). A comparative analysis of Genetic Programming. Chapter 2 of Advances in Genetic Programming 2, ed. P. J. Angeline and K. E. Kinnear, MIT Press.

### DESCRIPTION OF THE FIGURES

FIG. 1 is the diagram representation of a conventional mathematical expression, a LISP S-expression, and a coding region of a chromosome of the present invention.

FIG. 2 is the flowchart of the algorithm of the present invention.

FIG. 3 shows the structural organization of a chromosome.

FIG. 4 shows the expression of a chromosome as an expression tree.

FIG. 5 shows the mechanism of mutation.

FIG. 6 shows the mechanism of transposition.

FIG. 7 shows the mechanism of insertion.

FIG. 8 shows the mechanism of gene transposition.

FIG. 9 shows the mechanism of one-point recombination.

FIG. 10a e 10b show the mechanism of two-point recombination.

FIG. 11 is an initial population of 30 randomly generated chromosomes created to solve the problem of symbolic regression.

FIG. 12 is the best individual of the initial population for the problem of symbolic regression.

FIG. 13 is the perfect solution for the problem of symbolic regression.

FIG. 14 is the comparison between the present invention and genetic programming in the problem of symbolic regression.

FIG. 15 is an initial population of 30 randomly generated chromosomes created to solve the block stacking problem and the respective fitnesses for the particular set of initial states.

FIG. 16 is the first useful program discovered while solving the block stacking problem which removes all the blocks from the stacks.

FIG. 17 is the second useful program discovered while solving the block stacking problem which fills in partially all the stacks.

FIG. 18 is the correct solution for the block stacking problem which stacks completely and correctly all the stacks.

FIG. 20 is the best individual of the initial population for the problem of the multiplexer of 6 bits.

FIG. 19 is the comparison between the present invention and genetic programming in the block stacking problem.

FIG. 21 is a program discovered while solving the problem of the multiplexer of 6 bits that decodes correctly one address.

FIG. 22 is a program discovered while solving the problem of the multiplexer of 6 bits that decodes correctly two addresses.

FIG. 23 is a program discovered while solving the problem of the multiplexer of 6 bits that decodes correctly three addresses.

FIG. 24 is the correct solution for the problem of the multiplexer of 6 bits that decodes correctly four addresses.

### DETAILED DESCRIPTION OF THE INVENTION

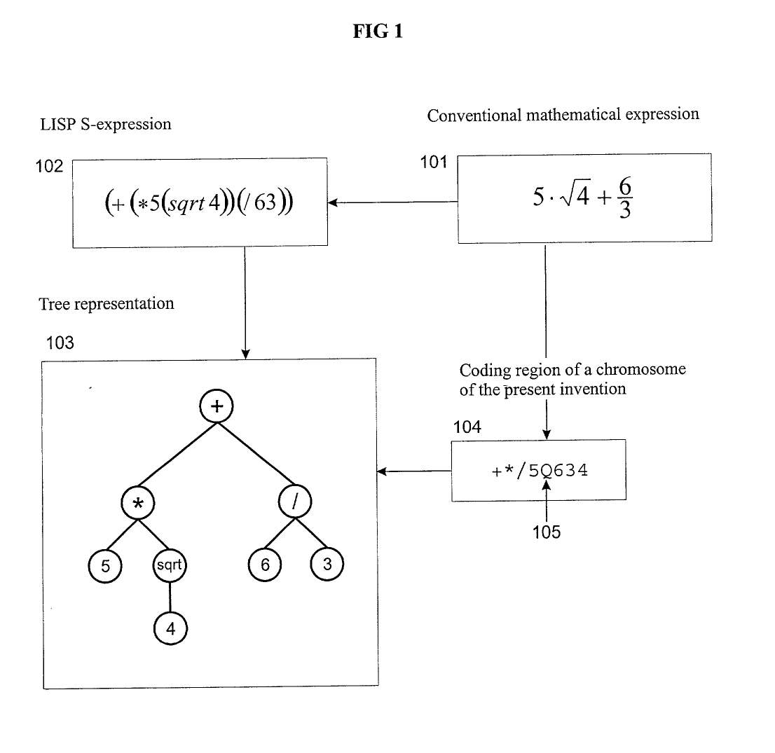

The non-linear entities created by genetic programming are diagram representations of LISP S-expressions. FIG. 1 shows a conventional mathematical expression 101; the correspondent LISP S-expression 102; the respective tree diagram representation 103; and its representation in a chromosome (coding region 104) of the present invention. The symbol ‘Q’ 105 in the coding region 104 of a chromosome represents the square root function.

Genetic programming creates initial populations of parse trees like the one shown in FIG. 1 (103), and these are the entities which are reproduced, recombined, permuted or, rarely, mutated (the genetic operators used by genetic programming). Nevertheless, these genetic manipulations are extremely complicated and problematic in this system, as the substitution of one argument by a function or vice versa, or the substitution of a function of two arguments by a function of one argument, like, for instance, the substitution of ‘*’ by ‘sqrt’ in FIG. 1, makes the parse tree and the correspondent S-expression invalid. The same problem appears in permutation where certain nodes are permuted. Therefore, genetic programming uses recombination almost exclusively as LISP permits this kind of modification. Thus, it is not easy for genetic programming to introduce variation in the population, which is the material of evolution. A way of solving this problem consists in the creation of huge initial populations where all the entities are different in order to discover, with a certain probability, a solution for the problem at hand, by means only of recombination of the material created in the initial population. This is one of the reasons why genetic programming is extremely expensive and inefficient.

Due to the invention of the genotype and the phenotype, the present invention allows the use, without restrictions, of several genetic operators, like for instance the genetic operators of the present invention: mutation, transposition, insertion, gene transposition, one-point recombination, and two-point recombination.

The present invention shares with genetic programming an identical form of tree representation, but the expressions that encode the expression trees of the present invention are not LISP S-expressions: they are expressions with a unique structural and functional organization developed by myself for the purposes of the present invention.

Indeed, this organization is the keystone of the present work and these expressions are, in fact, the genome of the individuals of the present invention.

This description proceeds with the detailed and generalized description of the algorithm of the present invention, presenting also three specific examples of applications of the present invention. The chosen examples show how the present invention can be applied to problems of symbolic regression, planning (block stacking), and design of electronic circuits (multiplexer of 6 bits).

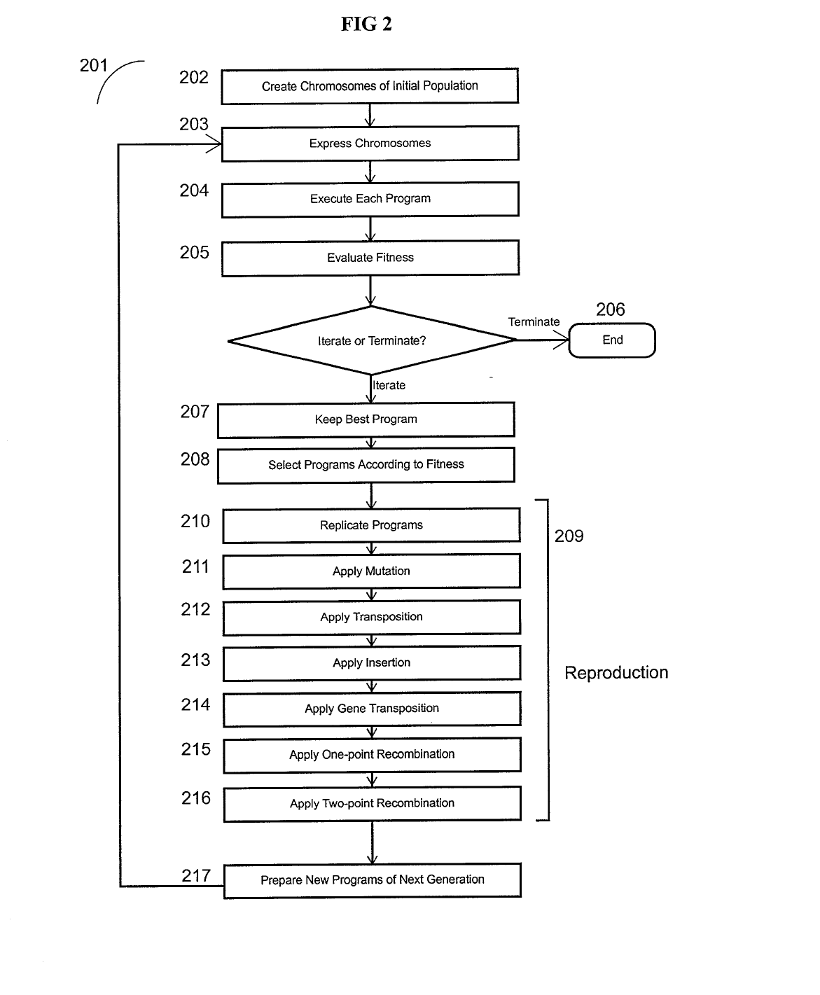

The flowchart of an algorithm of the present invention is shown in FIG. 2. The process 201 starts with the step 202 “Create chromosomes of initial population” where a certain number of chromosomes is randomly generated. The fundamental iterative loop of the process starts with the step 203 “Express chromosomes” where the language of the chromosomes is translated to the language of the expression trees. With the step 204 “Execute each program”, each program is executed, being the result of its performance evaluated in the step 205 “Evaluate fitness”. After that, the process is checked in order to determine if a solution has been found or if some other termination condition has been satisfied. If the termination condition was satisfied, the process terminates at “End” 206; otherwise the process continues.

With step 207 “Keep best program”, the program with highest fitness is chosen to reproduce without modification. In the next step 208 “Select programs according to fitness”, the programs are selected by fitness-proportionate selection, meaning that individuals with higher fitness have higher probability of leaving more offspring. Furthermore, in the present invention, the selection mechanism used involves a random factor, and sometimes the best individuals die without leaving offspring. This kind of selection is similar to natural selection and is usually used by different systems of evolutionary computation like genetic algorithms. Nevertheless, the present invention uses a simple kind of elitism (step 207), choosing the best individual of each generation to be reproduced without modification into the next generation (step 217).

The following seven steps consist of reproduction 209. In the step 210 “Replicate programs”, the chromosomes are copied to be transmitted to the next generation. Replication alone does not introduce variation in the population: if reproduction consisted only of replication, the same individuals would be reproducing indefinitely and populations would become less and less diverse for some individuals would not be lucky during selection. Variation appears only with the action of the remaining genetic operators: step 211 “Apply mutation”, step 212 “Apply transposition”, step 213 “Apply insertion”, step 214 “Apply gene transposition”, step 215 “Apply one-point recombination”, and step 216 “Apply two-point recombination”. As shown in FIG. 2, these steps are sequentially applied, being all the chromosomes randomly chosen and subjected to the set of chosen operators. Thus, with the present invention it is unlikely the creation of offspring exactly like the parents, being therefore extremely inventive and extremely efficient in problem solving.

Thus, in the present invention a set of six operators capable of creating genetic variation is used. The set of operators chosen for a particular problem depends on the nature of the problem, and different combinations of the different operators are used for particular problems. The set of operators of the present invention is more than sufficient to create the genetic variation necessary for evolution to occur, however, other operators may be easily created like inter-chromosomal transposition, multiple point recombination, recombination between three or more parents, deletion, inversion, permutation, etc.

After the process of reproduction 209 is complete, the newly created programs constitute the chromosomes of the individuals of the next generation which are prepared in the step 217 “Prepare new programs of next generation”. The process is repeated with the return to step 203 “Express chromosomes”.

Below is given a more detailed analysis of the most important steps of process 201. During the creation of the initial population, the genome of all the individuals is randomly generated using the symbols of the functions and terminals (arguments) chosen to solve the problem at hand. The chromosomes are linear entities of fixed length, composed of one or more genes. The genes are organized structurally in a head and a tail. The head contains symbols that represent both functions and terminals, whereas the tail contains only terminals, functioning as a repository of terminals.

For each problem, the length of the head (h) is chosen, whereas the length of the tail (t) is calculated in order to guarantee that the individuals created are structurally and functionally correct programs. The length of the tail depends on the number of arguments of the function with more arguments (n), and is evaluated by:

t=h(n−1)+1

For instance, for the set of chosen functions {Q, +, −,*, /} (square root, addition, subtraction, multiplication, and division, respectively, taking ‘Q’ one argument and the remaining functions two arguments), n is equal to 2.

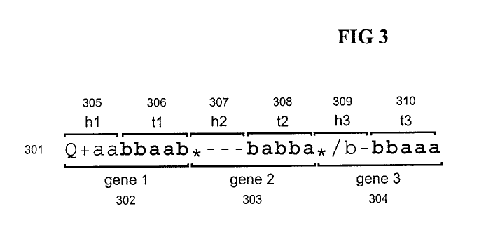

FIG. 3 represents a chromosome 301 with length 27, composed of 3 genes (302, 303, 304) where the head (305, 307, 309) is equal to 4 and the tail (306, 308, 310) is equal to 5. The symbols {Q, +, −, *, /} represent the chosen functions and the symbols {a, b} represent the chosen terminals to solve the problem at hand.

The chromosomes are afterwards expressed as expression trees, which are the entities that perform a certain task. The result of that task is associated to a value that determines selection.

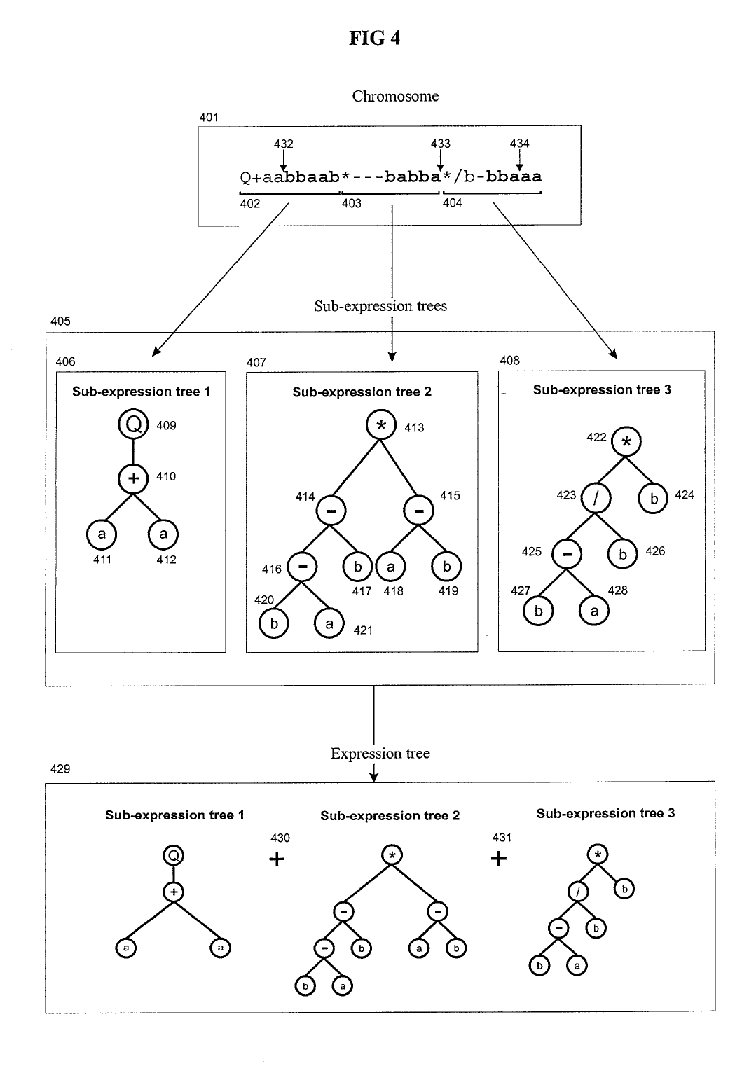

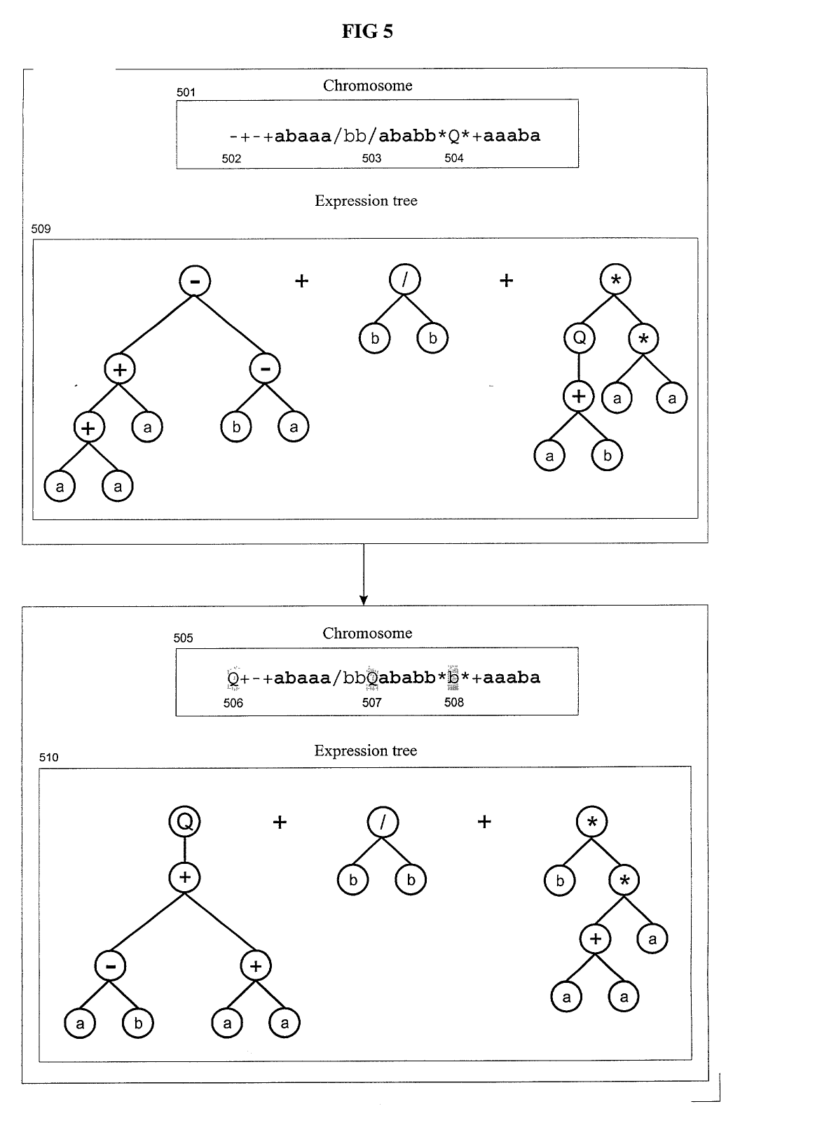

In FIG. 4 is shown how chromosome 401 is expressed in the respective expression tree 429. First, the individual genes (402, 403, 404) are expressed as sub-expression tress 405, being the expression straightforward and very simple: the first node (the root) of a sub-expression tree is the first symbol in the respective gene (‘Q’ 409 for gene 1 402; ‘*’ 413 for gene 2 403; ‘*’ 422 for gene 3 404); the second line of the sub-expression tree is formed attaching to that node as many branches as there are arguments to that function, being the circles filled with the characters of the gene, in the same order as they appear in the gene (for gene 1 402, ‘+’ 410 is the argument of ‘Q’ 409; for gene 2 40 , ‘−’ 414 and ‘−’ 415 are the arguments of ‘*’ 413; for gene 3 404, ‘/’ 423 and ‘b’ 424 are the arguments of ‘*’ 422); in the third line of the sub-expression tree are attached the arguments to the functions that appeared on the second line, being the circles filled with the next characters of the gene, in the same order as they appear in the gene (for gene 1 402, ‘a’ 411 and ‘a’ 412 are the arguments of ‘+’ 410; for gene 2 403, ‘−’ 416 and ‘b’ 417 are the arguments of ‘−’ 414 whereas ‘a’ 418 and ‘b’ 419 are the arguments of ‘−’ 415; for gene 3 404, ‘−’ 425 and ‘b’ 426 are the arguments of ‘/’ 423); in the fourth line of the sub-expression tree are attached the arguments to the functions that appeared on the second line, being the circles filled with the next characters of the gene, in the same order as they appear in the gene (for gene 1 402, the expression is complete, as the third line contains only terminals; for gene 2 403, ‘b’ 420 and ‘a’ 421 are the arguments of ‘−’ 416; for gene 3 404, ‘b’ 427 and ‘a’ 428 are the arguments of ‘−’ 425); this process is repeated for each gene until a base line containing only terminals is formed. In the case of FIG. 4, the first gene 402 is expressed in 3 lines (sub-expression tree 406) with the codifying sequence ending at termination point 432; gene 2 403 is expressed in 4 lines (sub-expression tree 407), with the terminal point 433 coinciding with the end of the gene; gene 3 404 is expressed in 4 lines (sub-expression tree 408) and ends at termination point 434. The sub-expression trees (406, 407, 408) are afterwards linked by a chosen function, in the case of FIG. 4 they are linked by addition (430 and 431). The linked sub-expression trees form the final expression tree 429 which produces the result that determines selection. Note that the linking functions (430 and 431) chosen to link the sub-expression trees (406, 407, 408) are not codified by the genome. This property of the present invention is similar to the posttranslational modifications that occur in nature like, for instance, the assembly of the subunits of a multimeric protein. Thus, the expression trees of the present invention have, like the parse trees of genetic programming, different sizes and shapes despite being codified by a linear chromosome of fixed length. It is also worth noticing the existence of genes in the present invention, as their use allows the discovery of simple blocks that are combined to form more complex structures, making the present invention a truly hierarchical invention system. The genetic programming technique is also known by hierarchical genetic algorithms, but some doubts remain about its hierarchical functioning. The programs of genetic programming consist of a single parse tree, and this greatly limits the discovery of simple blocks and their subsequent use in more complex programs. In fact, when the chromosomes of the present invention contain only one gene, the system discovers simple blocks and later uses them to form more complex individuals. But this single gene system is not as efficient as a multigenic one.

The multigenic system of the present invention allows the existence of neutral genes (genes that do nothing) which are fundamental for evolution to occur in the system. A neutral gene could be considered a gene which sub-expression tree returns a value that does not influence the final result of all the sub-expression trees codified by a chromosome. For instance, if the sub-expression trees were linked by addition, a neutral gene would code for a sub-expression tree that returns zero; in a Boolean problem where the sub-expression trees were linked by OR, a neutral gene would code for a sub-expression tree that returns zero. These genes are ideal targets for the accumulation of mutations, and they can be easily modified and transformed into a gene with expression.

As shown in FIG. 4, the different genes of a chromosome are expressed as sub-expression trees of different sizes and shapes, and, in most cases, not all the symbols of a gene are used to make a sub-expression tree. For instance, gene 1 402 in FIG. 4 with length 9 codes for the sub-expression tree 406 with 4 nodes (409, 410, 411, 412). The non-coding regions of a chromosome are also ideal targets for neutral mutations, as any mutation occurring downstream of the termination point (432, 434) of a gene has no effect in the product of expression of a gene and, therefore, are not subject to selection pressures. As in nature, these regions play an important role in evolution, as they can be easily activated by a genetic operator and integrated in a functional region of a gene.

It is worth noticing that the language of the chromosomes of the present invention is, per se, a new, simple and intuitive, programming language that can be used to program any computer. The operations that can be carried on in this system correspond to the mathematical and logical operators used by any conventional computer language, as well as other more sophisticated operators like the actions ‘A’ (do until true), ‘R’ (remove from stack), and ‘C’ (move to stack) created to solve the block stacking problem presented in this document.

Another important feature of the present invention, is the fact that the organization of the chromosomes allows their modification by any genetic operator, producing always syntactically correct programs. Below are shown the effects and mechanisms of the different genetic operators of the present invention.

After fitness-proportionate selection, the individuals are reproduced. As in nature, during reproduction the genomes of the individuals are subjected to several modifications which are the result of mutation, transposition, insertion, recombination and other genetic operators, creating the genetic variation fundamental for evolution to occur. In the present invention, the chromosomes are subjected to one or several genetic operators, creating the genetic variation necessary for solution finding.

The mutation operator changes any symbol on the chromosome into another, with the exception of the tails where a terminal can mutate only into another terminal. This way the structural organization of the chromosome is maintained and all the individuals created are structurally and functionally correct.

In FIG. 5 is shown the mechanism of mutation. Suppose that in chromosome 501, a mutation changed the function ‘−’ 502 in gene 1 to ‘Q’ 506; the function ‘/’ 503 in gene 2 to ‘Q’ 507; and the function ‘Q’ 504 in gene 3 to ‘b’ 508. The comparison of the expression trees before (509) and after (510) mutation shows how deep can be the effect of mutation in this system. It is worth noticing that the substitution of ‘/’ 503 by ‘Q’ 507 is an example of a neutral mutation. The mutation rate is chosen in order to create the ideal genetic variation. Typically, a mutation rate equivalent to 2 point mutations per chromosome is used. Note, however, that in the present invention there are no constrains both in the kind of mutation and the number of mutations in a chromosome: in all cases the newly created individuals are syntactically correct programs.

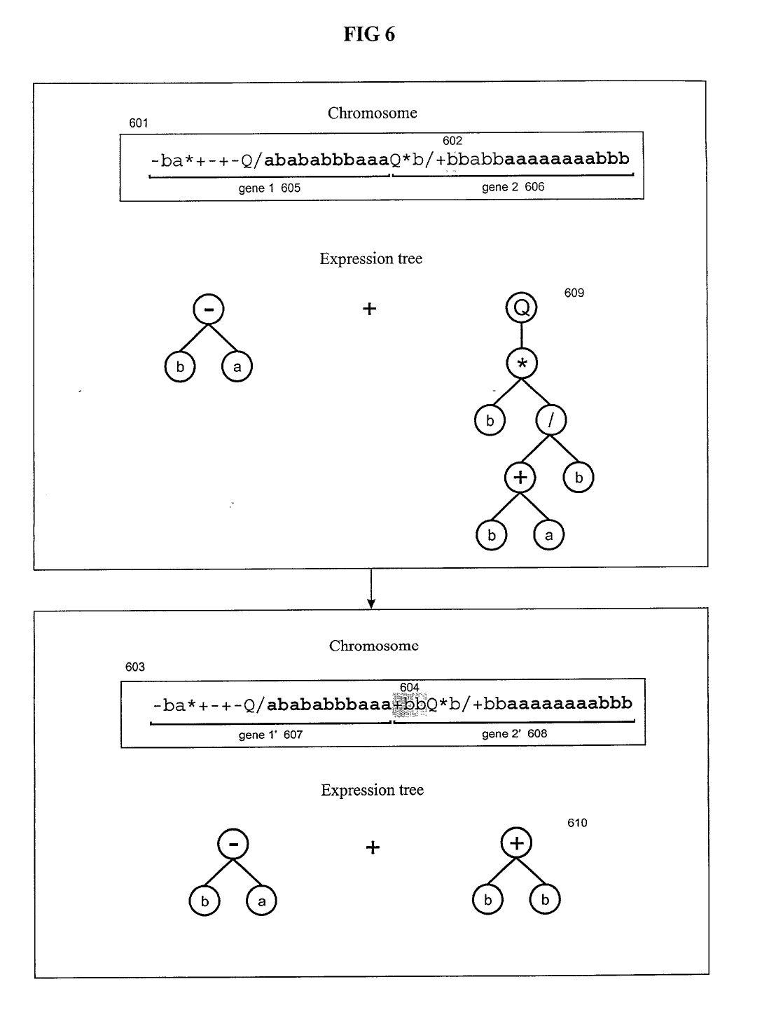

After mutation, the individuals are randomly chosen to undergo transposition, being, for each chromosome, also randomly chosen the gene to be modified by transposition. The intra-chromosomal transposition of transposable elements (transposons) is shown in FIG. 6. The transposons (602, 604), which may have different lengths, are chosen among the elements of the head and start always with a function. This kind of transposons jump to the beginning of genes. Consider the mechanism of transposition in a chromosome of length 42 (601) composed of two genes (605 and 606), each with length 21. Suppose that the sequence ‘+bb’ 602 of length 3 in gene 2 606 was chosen randomly to be a transposon. Then, a copy 604 of the transposon 602 is made into the beginning of the gene 2′608. Note that, during transposition, the whole head shifts to accommodate the transposon, losing, at the same time, its last symbols (as much as the length of the transposon). This way, the structural organization of the chromosome is maintained. Note also that the tail of the gene subjected to transposition and all nearby genes (gene 1′ 607) stay unchanged.

This kind of transposition allows the copy of small blocks and their propagation in the population. As with mutation presented above, transposition has a tremendous transforming power and is excellent to create genetic variation. Note that the sub-expression trees modified by this operator (sub-expression tree 609 before transposition and sub-expression tree 610 after transposition) are modified drastically, because the root itself is modified. This kind of operators prevent populations from becoming stuck in local optima, finding easily and rapidly good solutions.

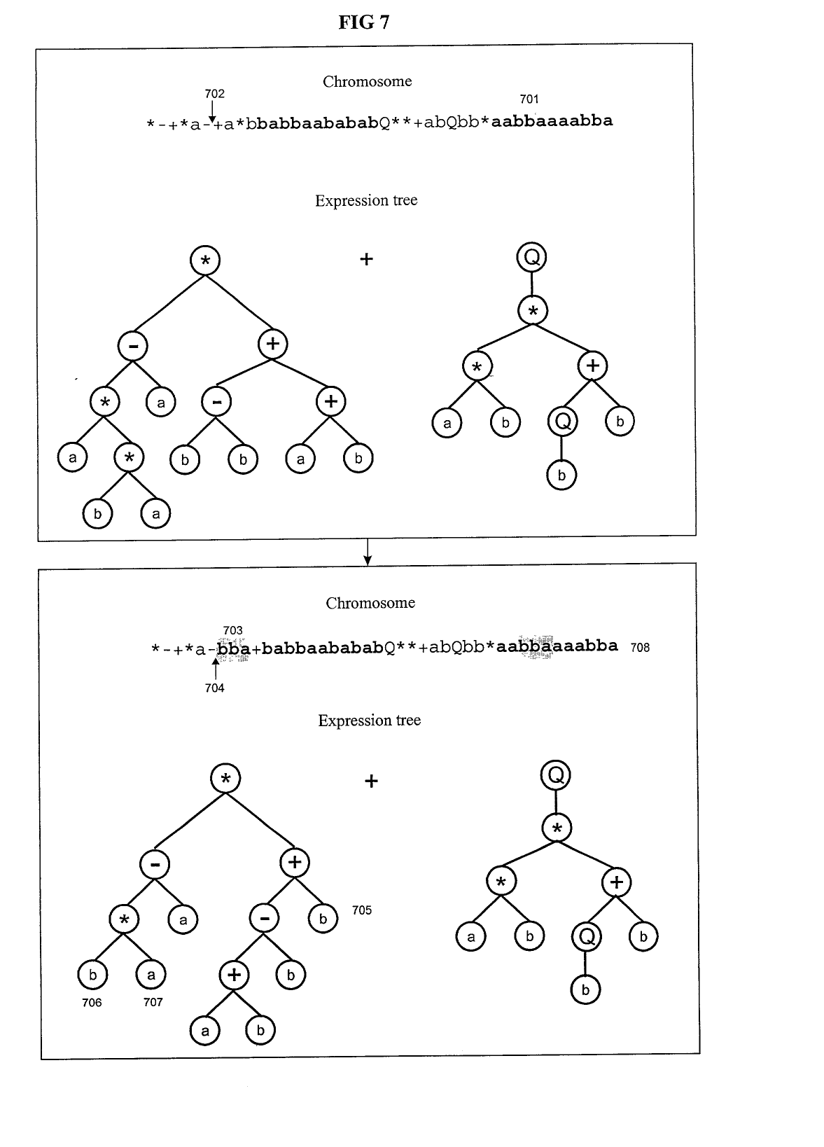

After transposition, some chromosomes are randomly chosen and subjected to intra-chromosomal insertion. For each chromosome subjected to insertion, one gene is also randomly chosen to be modified. Insertion is a more generalized case of transposition, where insertion elements of different lengths are chosen randomly throughout the chromosome and inserted anywhere in the head with the exception of the root. The insertion of an insertion element of length 3 is illustrated in FIG. 7. In this case, the insertion sequence element ‘bba’ 701 was chosen and inserted at the randomly chosen insertion site 702. This operator makes a copy 703 of the insertion sequence element 701 and inserts it at the insertion site 704; the sequence upstream the inserted element 703 stays unchanged, whereas the sequence downstream the inserted element 703 loses, at the end of the head, as many symbols as the length of the insertion element. This way, the structural organization of the chromosome is maintained.

It is worth noticing that when the newly created individual 708 is expressed, it is almost impossible to foresee which positions the symbols of the insertion element (705, 706, 707) will occupy, becoming most of the times separated and integrated in different functional blocks. This is similar to what happens during the folding of proteins in their three-dimensional structure, where amino acids encoded further apart in the DNA are brought together in the protein. Thus, as in nature, the present invention works blindly: the modifications that are made in the chromosomes are very different from the modifications a mathematician would make; nevertheless, they work very well. It is worth noticing that the genetic operators of genetic programming resemble more the logic and calculated work of a mathematician than the blind way of nature, recombining and permuting mathematically concise blocks.

As the operators above described, insertion is an excellent source of genetic diversity, forming new individuals capable of expressing new properties.

After insertion, some chromosomes are randomly chosen to be modified by gene transposition. Gene transposition is a special case of transposition where an entire gene (except the first) is spliced and transposed to the beginning of the chromosome.

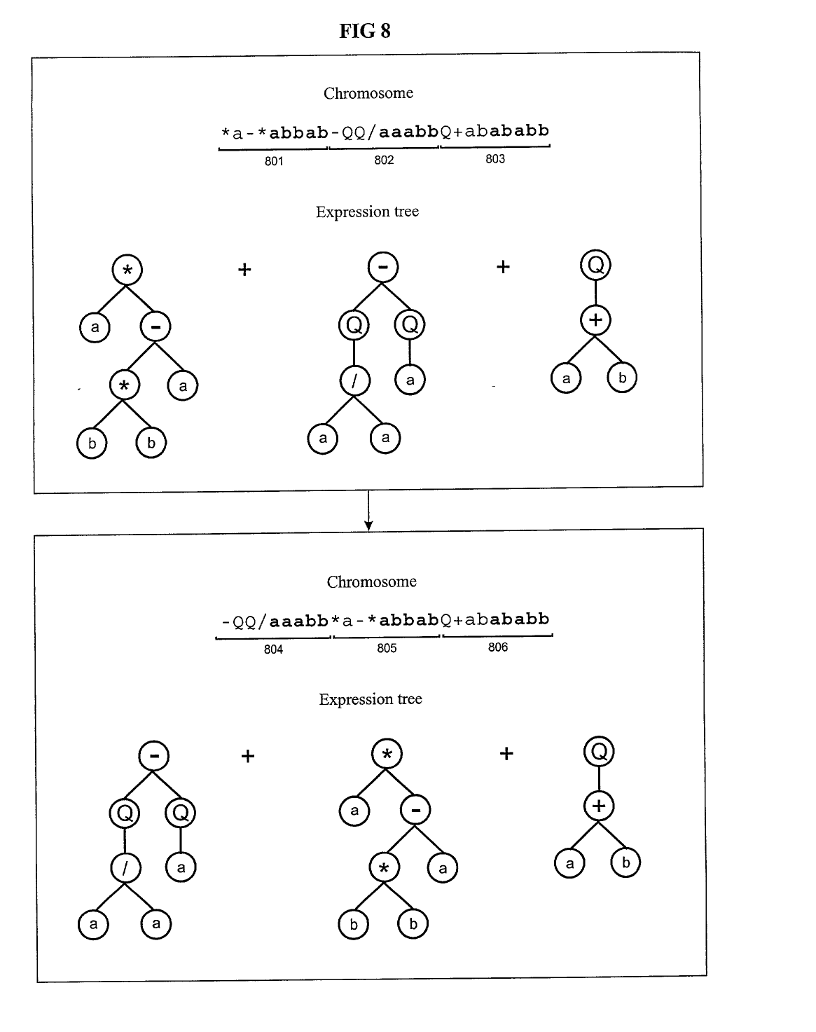

In FIG. 8 is shown the mechanism of gene transposition. In this case, gene 2 802 is transposed to the beginning of the chromosome: gene 2 802 becomes the first (804), gene 1 801 becomes the second (805) and gene 3 803 occupies the same position (806).

Note that for numerical applications where the function chosen to link the genes is addition (as in FIG. 8), the expression evaluated by the chromosome is not modified. But the situation differs in other applications where the linking function is not commutative, for instance, the Boolean function if (x,y,z) (if x=1, return y; otherwise return z). In this case the newly created individual is not equivalent to the parent.

Nevertheless, the transforming power of gene transposition reveals itself when this operator is conjugated with other operators, like one-point or two-point recombination. For example, if two functionally identical chromosomes or two chromosomes with an identical gene in different positions recombine, a new individual with a duplicated gene might appear. It is know that the duplication of genes plays an important role in biology and evolution, and, in fact, in the present invention, individuals with duplicated genes are commonly found in the process of problem solving.

After gene transposition, pairs of chromosomes are randomly chosen to undergo one-point recombination. During one-point recombination, the two parent chromosomes are paired and exchange some material between them.

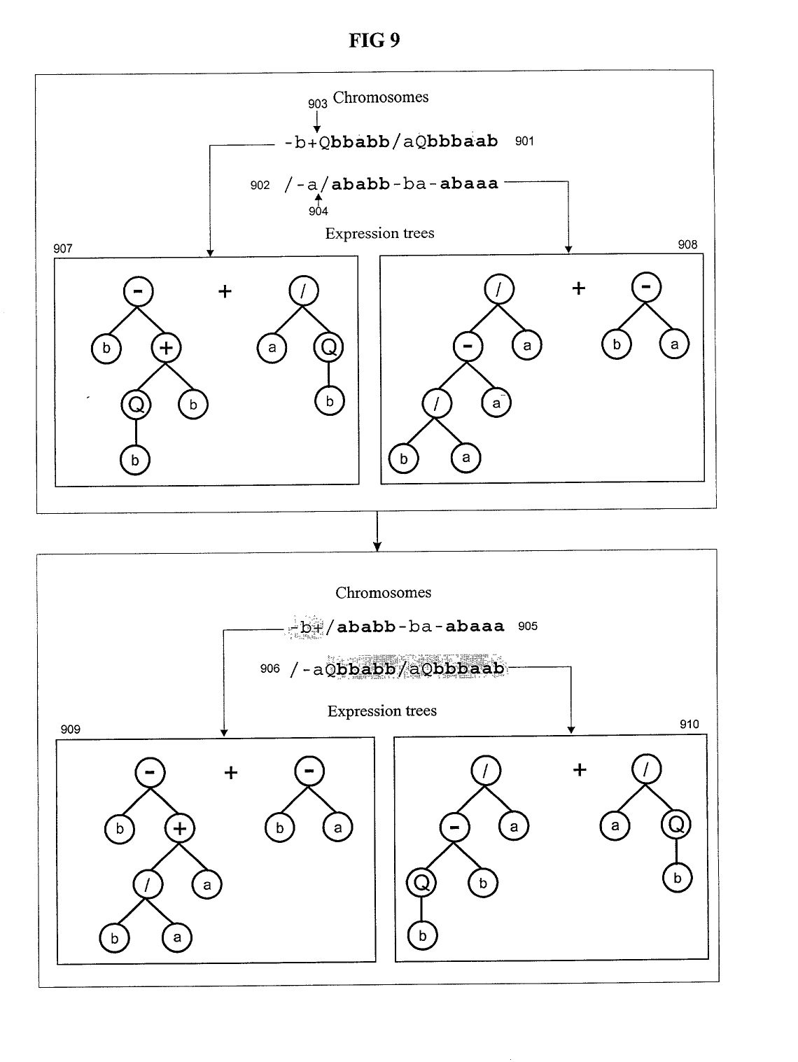

In FIG. 9 is shown the mechanism of one-point recombination between two chromosomes (901, 902) of length 18, composed of two genes. The recombination point (903, 904) is randomly chosen and the paired chromosomes are cut at the recombination point (903, 904), exchanging between them the fragments downstream the recombination point. With this kind of recombination, most of the times, the daughter chromosomes created (905, 906) are not only different from one another but are also different from the parents (901, 902). Note that, in the case of FIG. 9, the expression trees of the parents (907, 908) and the expression trees of the newly created individuals (909, 910) are all different.

Thus, one-point recombination, like the above mentioned operators, is a very important source of genetic variation, being, after mutation, the genetic operator most chosen in the present invention.

After one-point recombination some chromosomes are randomly chosen to undergo two-point recombination.

The kind of two-point recombination of the present invention was implemented to allow the exchange of complete genes. These genes occupy the same position in the parent chromosomes. Thus, with this kind of recombination the parent chromosomes are paired and a gene randomly chosen is exchanged between the parents.

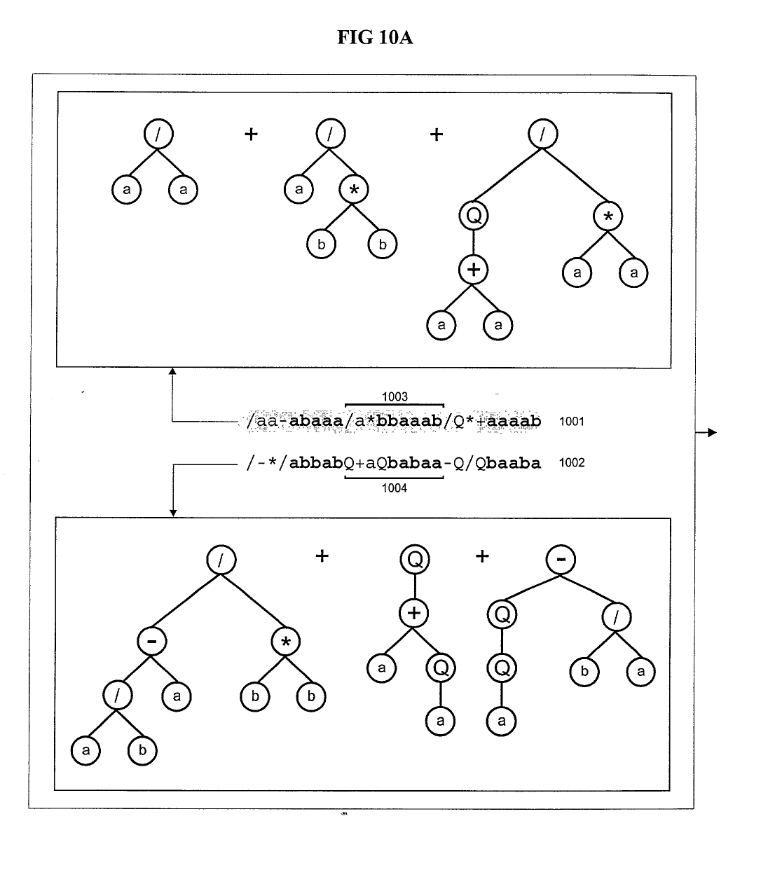

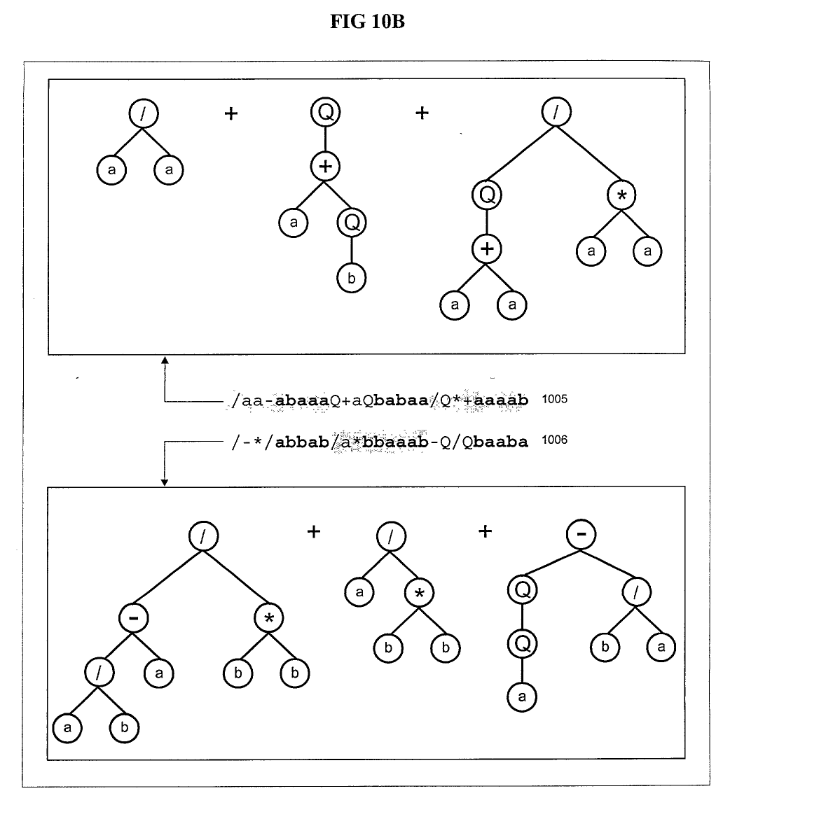

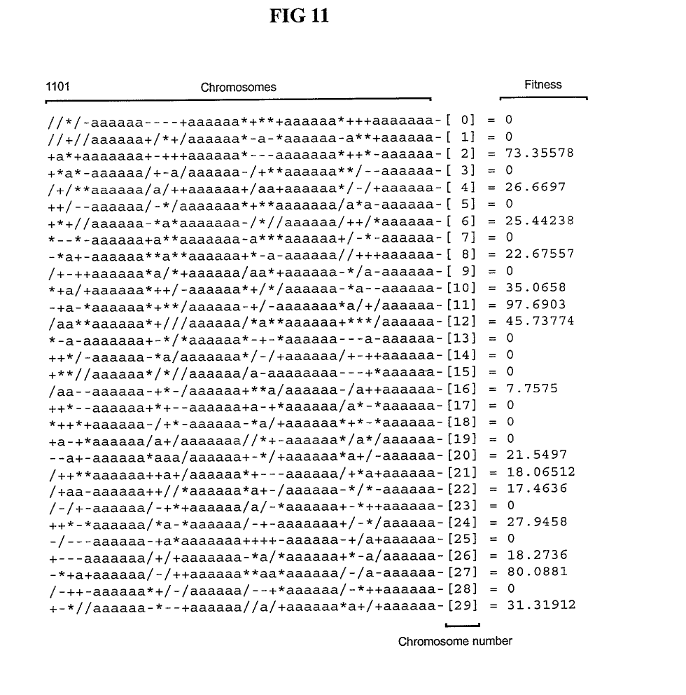

In FIG. 10a and 10b is shown the two-point recombination between two chromosomes (1001, 1002) composed of three genes. In this case, gene 2 (1003, 1004) was chosen to be exchanged between the parent chromosomes, being the chromosomes cut by the bonds that delimit the gene. As a result, two new individuals (1005, 1006) are formed, with chromosomes containing genes from both parents.

Note that, as in one-point recombination, the newly created individuals differ, most of the times, both between themselves and the parents. Two-point recombination is also an important source of genetic variation, being, together with one-point recombination, one of the operators most frequently chosen in the present invention.

It is worth noticing that in the present invention, the number and type of genetic operators are chosen by the user, being used, most of the times, a combination of two or more genetic operators to create genetic variation in the population and, therefore, guarantee the discovery of a good solution to the problem at hand. However, and in contrast to genetic programming, all the chromosomes modified by the operators of the present invention are randomly chosen and therefore one chromosome could be chosen to be modified by none or several operators at a time, accumulating different transformations. This makes the present invention extremely creative and efficient in the discovery of solutions. In fact, this is one of the reasons that allows the present invention to find solutions using, for the same problems, population sizes that are usually more than one order of magnitude inferior to the ones used by genetic programming (for instance, for the symbolic regression problem presented here, genetic programming uses population sizes of 500 entities whereas the present invention uses population sizes of only 30 individuals; see also the other examples presented in this document).

Another important difference between the present invention and genetic programming consists in the set of genetic operators used by both systems and their implementation. Genetic programming uses almost exclusively a tree level one-point recombination, using mutation very rarely. Furthermore, in genetic programming, the entities are either selected to recombine or mutate, never being subjected to more than one operator at a time in one reproductive cycle. These are additional reasons that force genetic programming to use huge population sizes, as the genetic diversity must be already present among the entities of the initial population (in fact, genetic programming uses lots of computational resources in order to guarantee that all the entities of the initial population are different from one another); if mutation is not being used, it is only by recombining the blocks already present in the initial population that genetic programming discovers solutions. Only by using huge population sizes is genetic programming capable of guaranteeing with a certain probability that all the elements necessary for the discovery of a solution were already present in the initial population.

On the other hand, in genetic programming, the entities subjected to a particular operator are carefully chosen, making that technique extremely expensive in terms of computational resources.

In the present invention, the reproductive cycle is complete after two-point recombination, and the newly created chromosomes consist of the genomes of the individuals of the next generation. These individuals are, in their turn, subjected to the same developmental process: expression of the genomes as expression trees, confrontation of the selection environment, and reproduction.

Below follow three examples chosen from different fields in order to illustrate the use of the present invention: symbolic regression or function finding, block stacking and the multiplexer of 6 bits.

Symbolic Regression or Function Finding

The objective of this problem is the discovery of a symbolic expression that satisfies a set of fitness cases. The set of fitness cases consist of the selection environment where the adaptation of the individuals occurs.

The following function was chosen to illustrate this problem:

y=a+a+a+a

This function was chosen because it exhibits already a certain complexity and because it was used by J. Koza, therefore allowing the comparison of the present invention with genetic programming.

For problems of this complexity, the present invention requires usually a set of 10 fitness cases (the input) randomly chosen over a certain interval, for instance between −10 and 10. In this case, the goal is to find a function fitting those values within 0.01 of the correct value.

The function set chosen for this problem consisted of {+, −, *, /} and the terminal set consisted of the independent variable {a}. An initial population of 30 random chromosomes composed of 4 genes of length 11 was generated using the set of chosen functions and terminals. The chromosomes were expressed and their fitness determined against the set of fitness cases. In this case, for each fitness case, the fitness was evaluated by the expression:

f=M−|E|

where M is the selection range and E the absolute error between the number generated by the expression tree and the target value. The selection range is chosen for each problem, being in this case 100. Then, if E is less or equal to 0.01 (the chosen precision), f=100. Thus, if the 10 fitness cases were computed exactly or within the 0.01 of the target value, the maximum fitness (fₘₐₓ) would be 1000.

In FIG. 11 is shown an initial population 1101 of randomly generated chromosomes created in one experiment. Note that 15 of the individuals randomly generated in the initial population are fit to solve partially the problem at hand. For instance, the best individual of this generation has f=97.6903, meaning that none of the individuals of this population is capable of solving even one fitness case within the chosen precision (0.01). However, as will be shown, these cumbersome individuals are capable of leaving descendants 100% fit to solve the problem.

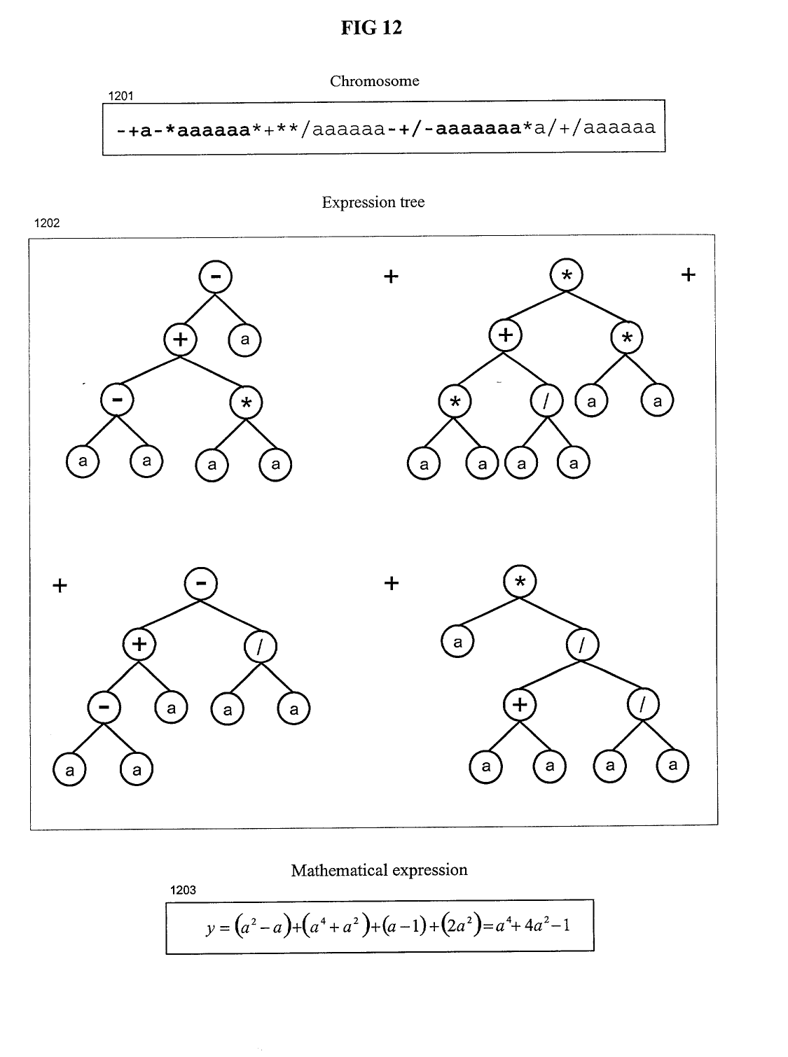

The chromosome of the best individual 1201 of the initial population, the respective expression tree 1202, and the correspondent mathematical expression 1203 are shown in FIG. 12. The brackets in the mathematical expression 1203 show the contribution of each sub-expression tree in order to simplify the analysis. Notice that, after simplification, only one of the terms (a⁴) of the mathematical expression 1203 coincides with the target function.

The fit individuals of the initial population are afterwards selected according to fitness, and are reproduced creating the individuals of the next generation. In this problem, the process was repeated for 50 generations or until a solution was found.

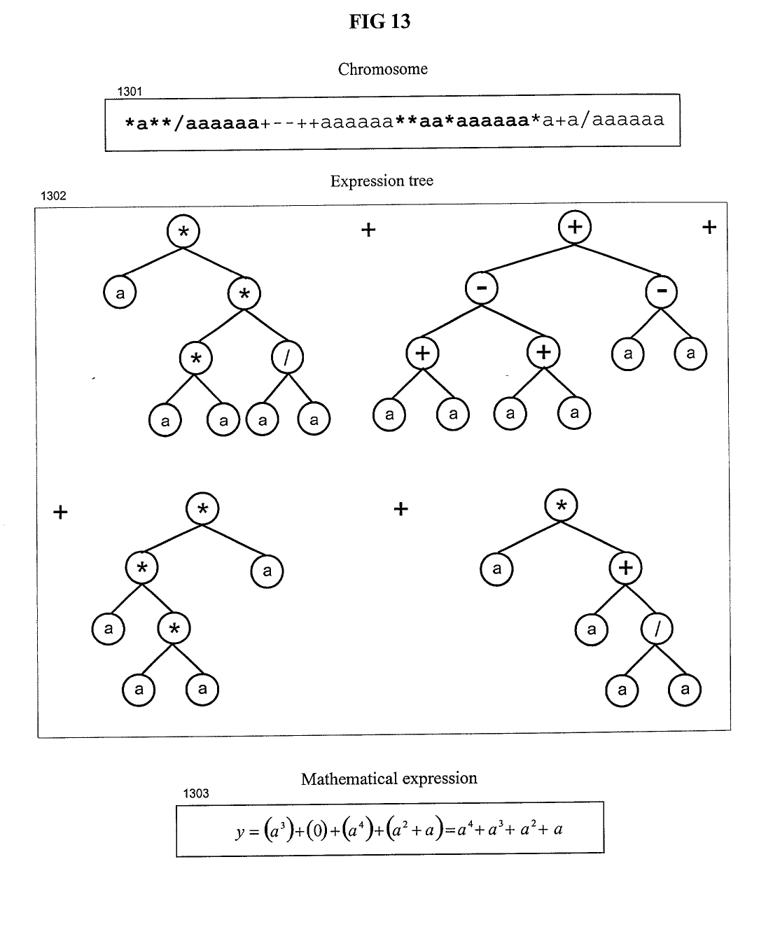

In FIG. 13 is shown the descendant 1301 of the successful individuals of the initial population. This descendant has maximum fitness 1000, being therefore capable of solving this problem correctly. This individual was created after 8 generations, and its expression tree 1302 corresponds to a mathematical expression 1303 equivalent to the target function.

The probability of success for this problem was evaluated over 100 independent runs, having the maximum value of 1, i.e. in all the runs a perfect solution was found. The comparison of the performance of the present invention with that of genetic programming shows that the present invention surpasses genetic programming in 374 times.

The measure used to compare both systems is usually used to compare different evolutionary systems and depends on the number of fitness functions evaluation necessary to find a correct program with a certain probability.

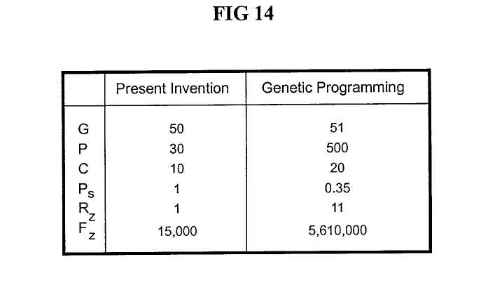

The comparison of the present invention with genetic programming for 100 independent runs is shown in FIG. 14.

The number of independent runs R_{z} required to find a correct solution by generation G with a probability z of 0.99 is evaluated by the formula:

$${R}_{z} = \frac{log ( 1 - z )}{log ( 1 - {P}_{s} )} being {P}_{s} \neq 1$$

where Pₛ is the probability of success; if Pₛ=1, then R_{z=}1.

The number of fitness-functions evaluations F_{z} needed to find a correct program with a certain probability z=0.99 is evaluated by the formula:

F_{z}=G·P·C·R_{z}

where G is the number of generations; P the population size; and C the number of fitness cases.

Thus, the comparison of F_{z} values obtained by the present invention and genetic programming for this problem (FIG. 14) shows that the present invention surpasses genetic programming in 374 times (5,610,000/15,000), more than two orders of magnitude.

Block Stacking

This toy problem is a planning problem frequently used in artificial intelligence and it is considered a sophisticated problem.

In this problem the input is a set of initial configurations of blocks (for instance, the letters of the word ‘universal’) randomly distributed between the stack and the table. The blocks on the table are all accessible whereas in the stack only the top block is accessible, and it is only possible to remove this block or put another block on the top of it.

In block stacking, the goal is to find a plan that takes any initial configuration of blocks randomly distributed between the stack and the table and places them in the stack in the correct order, i.e. as they appear in the word ‘universal’.

The functions and terminals used for this problem consisted of a set of actions and sensors. The set of actions consisted of 4 functions {C, R, N, A} (move to stack, remove from stack, not, and do until, true, respectively), where the first three take one argument and ‘A’ takes two arguments. The set of sensors consisted of 3 terminals {u, t , p} (current stack, top correct block, and next needed block, respectively). The top correct block ‘t’ refers only to the block on the top of the stack and whether it is correct or not; if the stack is empty or has some blocks, all of them correctly stacked, the sensor returns True, otherwise returns False. The next needed letter ‘p’ refers obviously to the next needed block immediately after ‘t’.

All the problems involving an iterative action like ‘A’ (do until true) raise some problems due to the memory resources available in the computer. Therefore it is necessary to establish rules concerning the functioning of these actions. Thus, in the present invention, the ‘A’ loops are processed at the beginning, are solved in a particular order (from bottom to top and from left to right), the action argument is executed at least once despite the state of the predicate argument, and each loop is executed only once, timing out after 20 iterations.

The fitness was determined against 10 fitness cases (initial configurations of blocks). Each generation, an empty stack plus 9 initial configurations with one to nine letters in the stack were randomly generated. The empty stack was used to prevent the untimely termination of runs, as a fitness point was attributed to each empty stack (see below). However, the present invention is capable of solving this problem efficiently, using uniquely 10 random initial configurations. This, in fact, distinguishes the present invention from genetic programming as the later technique is only capable of solving this problem using 167 fitness cases, cleverly constructed to cover the various classes of possible initial configurations (10 fitness cases with 0-9 letters correctly stacked and the remaining on the table; nine fitness cases with 0-7 letters correctly stacked and exactly one letter incorrectly on top, with the remaining letters on the table; and 148 random fitness cases).

An initial population of 30 random chromosomes composed of 3 genes of length 9 was generated using the set of chosen functions and terminals. The sub-expression trees (sub-plans) were executed sequentially, for instance, if the first sub-plan empties all the stacks, the second sub-plan may proceed to fill them partially, and the third may proceed to fill them completely. To make this clear, the expression trees are linked by ‘+’, representing exclusively the sequential order in which the sub-plans are executed; like all linking symbols used to link the sub-expression trees, this ‘+’ is extra-chromosomal. The chromosomes were afterwards expressed and their fitness was determined against the selection environment of the 10 above described fitness cases. The fitness function was as follows: for each empty stack one fitness point was attributed; for each partially and correctly packed stack two fitness points were attributed; and for each completely and correctly filled stack 3 fitness points were attributed. Thus, the maximum fitness was 30. The idea was to make the population of programs hierarchically evolve solutions toward a perfect plan. And, in fact, first a plan was discovered that empties all the stacks, then some programs learned how to partially fill those empty stacks, and finally a perfect plan was discovered that fills the stacks completely and correctly.

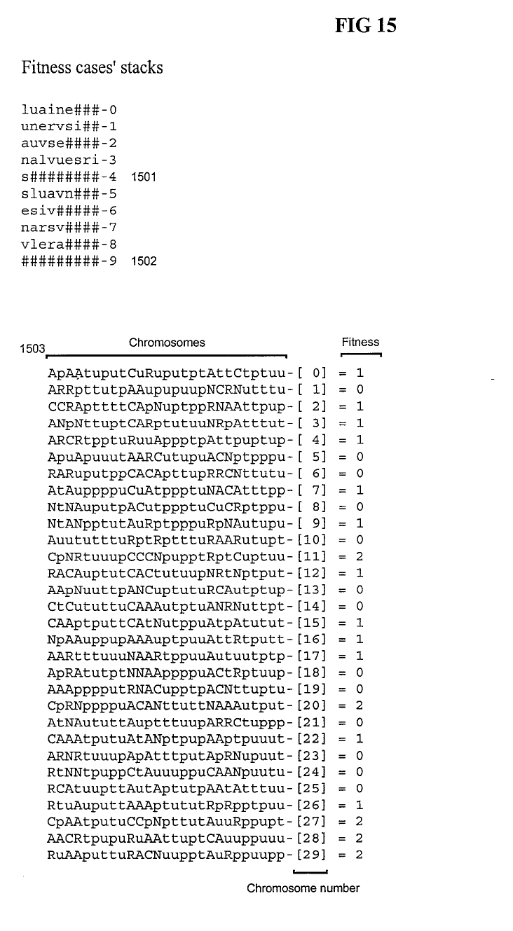

In FIG. 15, an initial population 1503 of random chromosomes created in one experiment is shown. Note that of the individuals created in the initial population, 17 have positive fitness. However, not a plan appeared in the initial population capable of doing anything useful, having the viable individuals a fitness of 1 or 2, meaning that they do nothing and have a fitness point due to the empty stack 9 (1502) or else they can remove a letter from the stack, receiving, for the particular case of initial configurations, two fitness points: 1 for the empty stack 9 (1502) and another for the initial configuration 4 (1501) which had only a letter and became empty after the letter was removed.

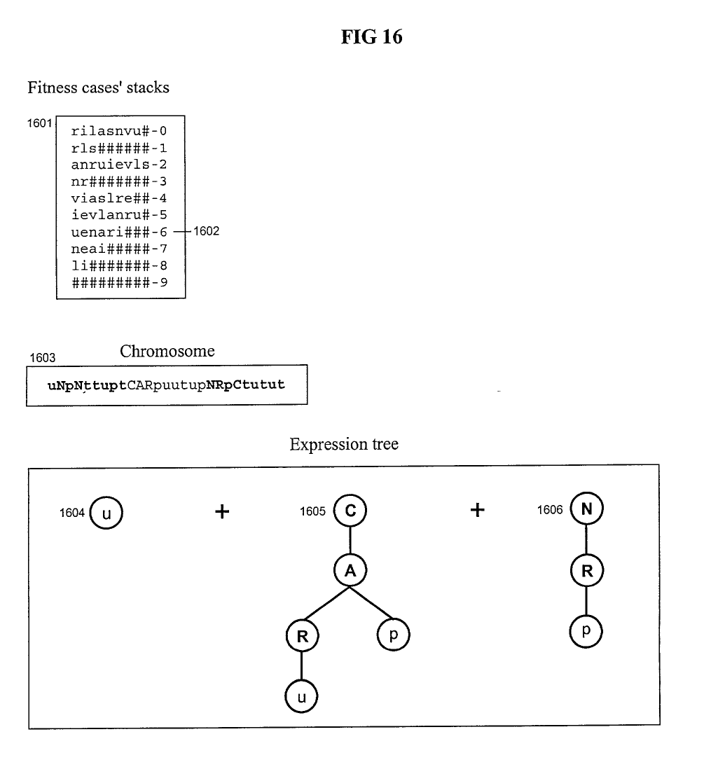

In the next generation the first useful program (1603) was discovered (FIG. 16). This plan removes all the letters incorrectly stacked, receiving a total of 11 points for the particular set of initial configurations 1601: 2 points for stack 6 1602 that stays with a correct letter after the incorrect letters were removed, more 9 points for the remaining stacks that were emptied. Note that the first sub-expression tree 1604 and the last 1606 contribute nothing to the problem at hand, being all the work done by the second sub-expression tree 1605.

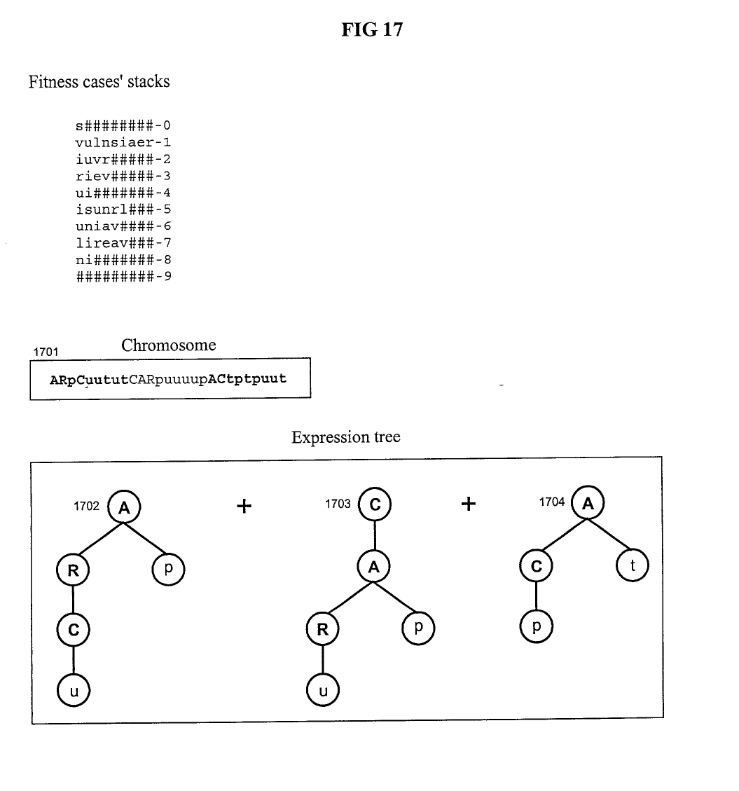

In generation 4 a more sophisticated plan (1701) was discovered (FIG. 17). This plan not only is capable of removing all the incorrectly stacked letters but also is capable of putting in all the stacks a correct letter, receiving a total of 20 fitness points: 2 points for each partially and correctly filled stack. Note that the first sub-expression tree 1702 and the second 1703 are homologous, doing exactly the same. These sub-expression trees are both capable of removing all the letters incorrectly stacked. The last sub-expression tree 1704 proceeds by putting one correct letter in all the empty stacks or stacks already with one or more letters correctly stacked.

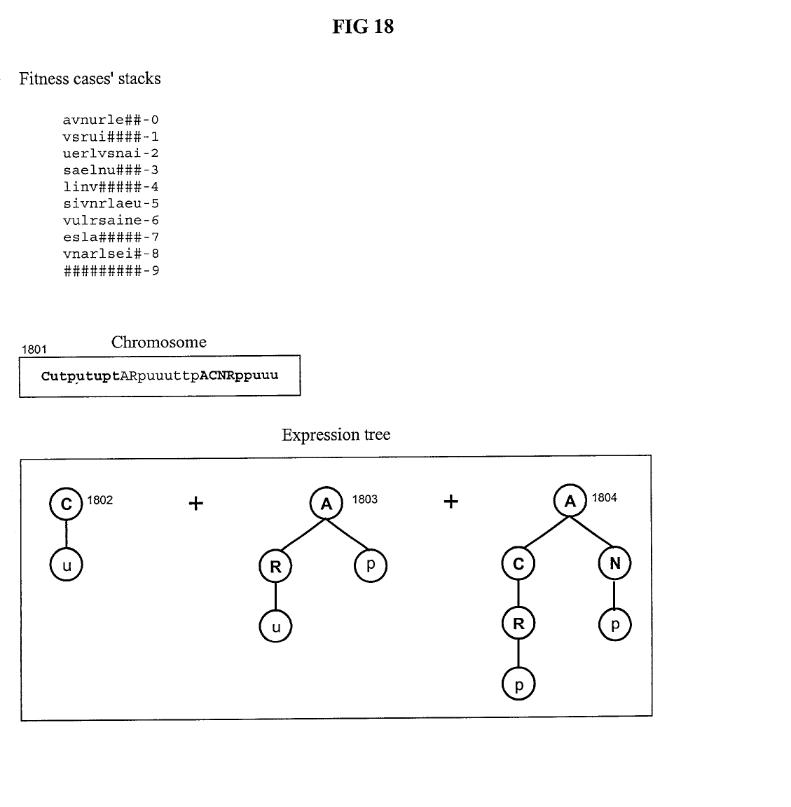

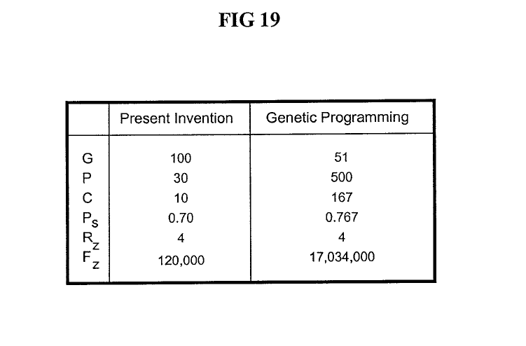

In generation 13 a perfect plan (1801) was found (FIG. 18). This plan starts by removing from the stack all the incorrect letters and proceeds by filling in all the stacks correctly and completely. This plan is a universal plan and its fitness has the maximum value of 30: three fitness points for each stack completely and correctly stacked. Note that the first sub-expression tree 1802 does nothing, being all the work done by the second sub-expression tree 1803 that removes all the incorrectly stacked letters and by the third sub-expression tree 1804 that fills the stacks with the remaining letters.

The probability of success for this problem was evaluated over 100 independent runs, being in this case 0.70. The comparison of the performance of the present invention with that of genetic programming shows that the present invention surpasses genetic programming in 142 times, despite the use by the present invention of 9 (out of 10) random initial configurations. This is very important, as in real life applications not always is possible to predict the kind of cases that would make the system discover a solution.

In FIG. 19 is shown the comparison for 100 independent runs of the present invention and 30 independent runs of genetic programming (see how to evaluate the performance in the symbolic regression problem above).

Thus, the comparison of F_{z} values obtained by the present invention and genetic programming for this problem (FIG. 19) shows that the present invention surpasses genetic programming in 142 times (17,034,000/120,000), more than two orders of magnitude.

The Multiplexer of 6 Bits The multiplexer of 6 bits is a logic circuit frequently used in the design of microprocessors and in telecommunications, allowing the serialization of parallel channels of communication.

The task of the 6-bit Boolean multiplexer is to decode a 2 binary address (00, 01, 10, 11) and return the value of the correspondent data register (d₀, d₁, d₂, d₃). Thus, the Boolean 6-multiplexer is a function of 6 activities: two, a₀ and a₁, determine the address, and four, d₀ to d₃, determine the answer. As the present invention uses character chromosomes, the terminal set consisted of {a, b, 1, 2, 3, 4} which correspond respectively to {a₀, a₁, d₀, d₁, d₂, d₃}.

There are 26=64 possible combinations for the 6 arguments and, in this case, the entire set of 64 combinations was used as the fitness cases for evaluating fitness. To determine the fitness, the 64 fitness cases were assembled in four sub-sets, each containing the 16 combinations correspondent to each address. The fitness of a program is the number of fitness cases where the Boolean value returned is correct, plus a bonus of 84 fitness points for each sub-set of combinations decoded correctly as a whole. Thus, for each decoded address a total of 100 fitness points were attributed and the maximum fitness was 400. The idea was to make the algorithm decode one address at a time. And, in fact, the algorithm learns to decode first one address, then another, until the last one.

The function set chosen for this problem consisted of the Boolean functions {A, O, N} (AND, OR, and NOT, respectively, taking the last function one argument and the remaining functions two arguments).

An initial population of 250 random chromosomes composed of 4 genes of length 11 was generated using the set of chosen functions and terminals. For this problem, the sub-expression trees were linked by the Boolean function OR. The chromosomes were expressed and their fitness evaluated against the set of 64 fitness cases.

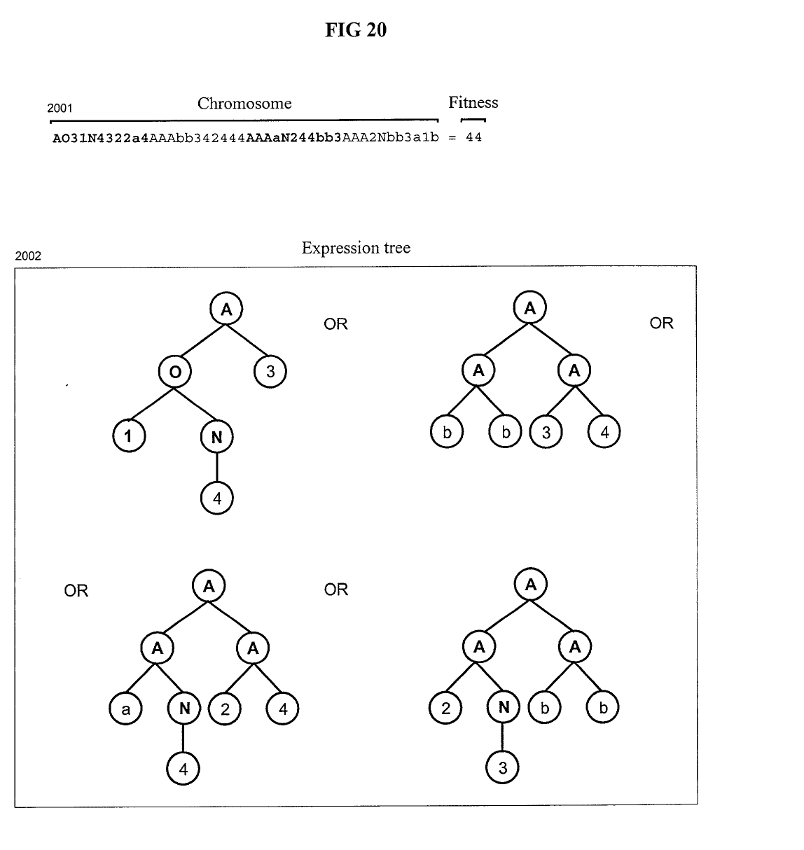

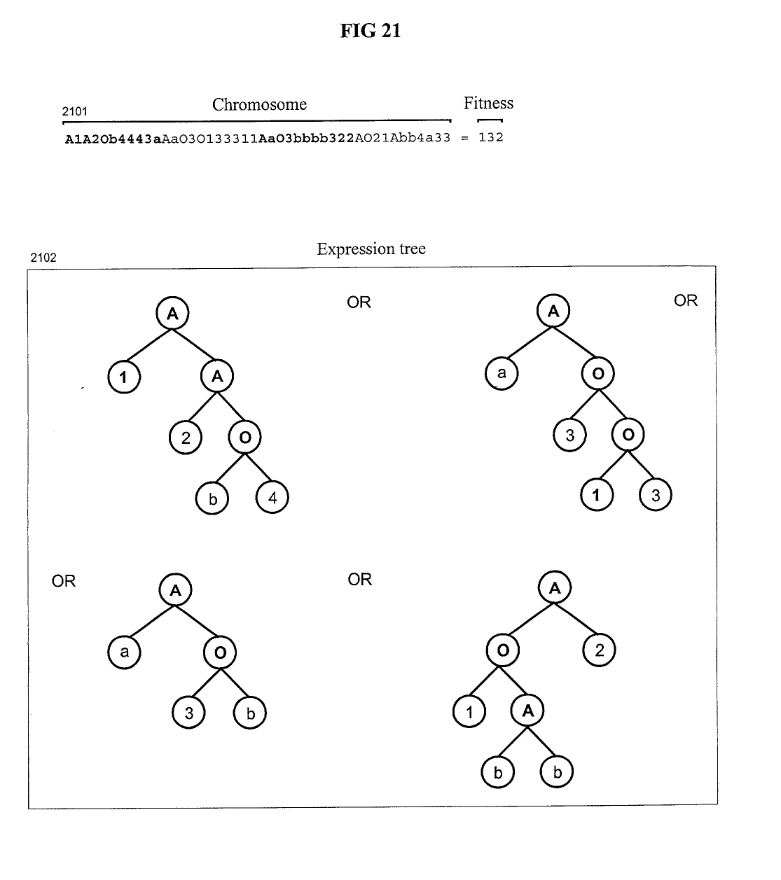

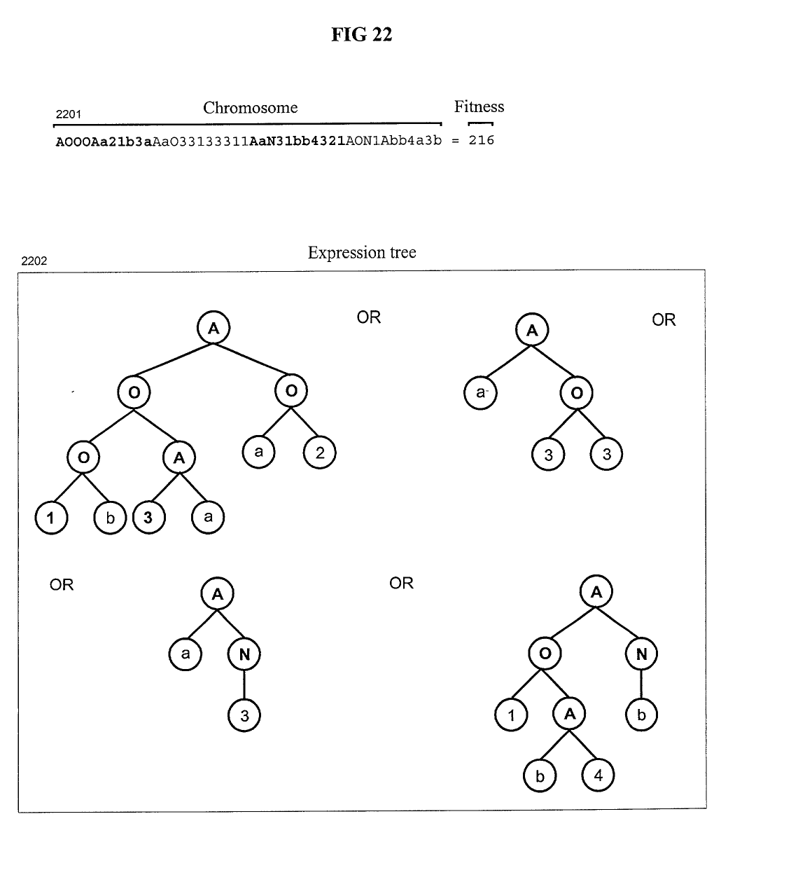

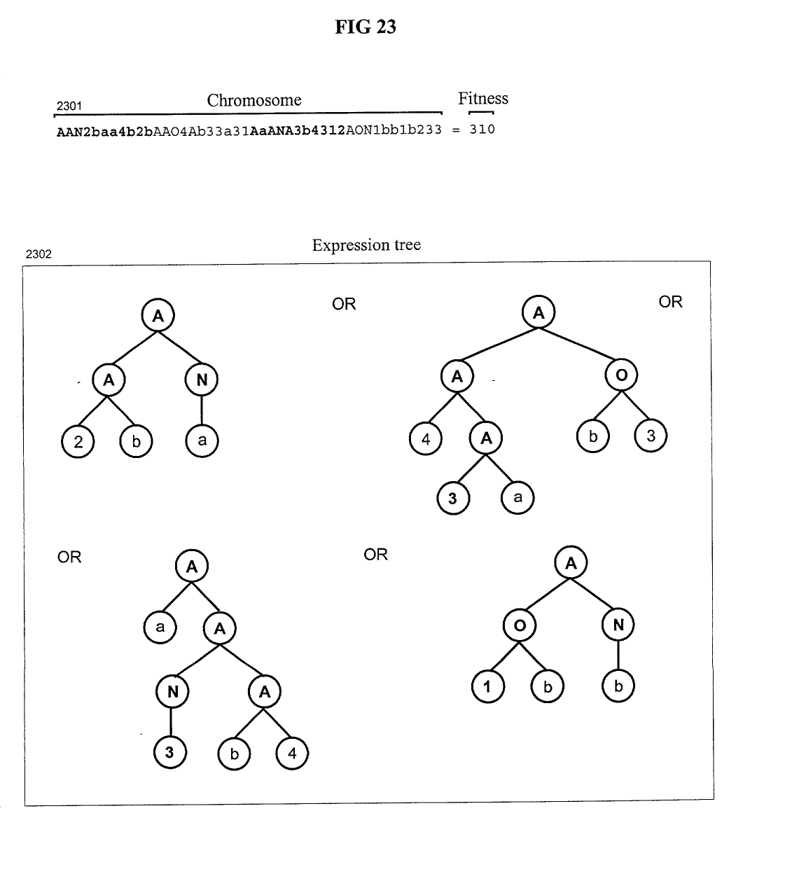

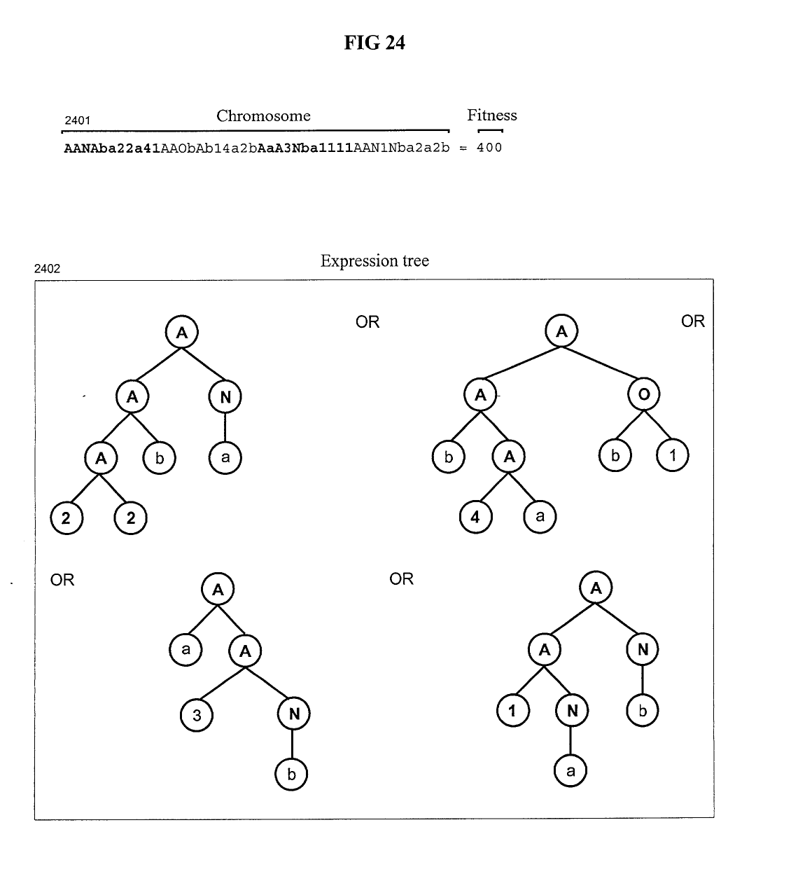

In FIG. 20 is shown the best individual 2001 of the initial population and the corresponding expression tree 2002. Note that this individual is capable of decoding 44 of the 64 fitness cases; however, it could not completely decode a single address, and therefore does not receive a fitness bonus.

The fit individuals of the initial population are afterwards selected according to fitness, and are reproduced creating the individuals of the next generation. In this problem, the process was repeated for 100 generations or until a solution was found.

In generation 4 an individual was created capable of decoding completely one address (16 fitness cases) more 32 fitness cases scattered throughout the remaining addresses, having a fitness of 132. The chromosome 2101 of this individual and the respective expression tree 2102 are shown in FIG. 21.

In generation 12 an individual was created capable of decoding completely two addresses (32 fitness cases) more 16 fitness cases scattered throughout the remaining addresses, having a fitness of 216. The chromosome 2201 of this individual and the respective expression tree 2202 are shown in FIG. 22.

In generation 27 an individual was created capable of decoding completely three address (48 fitness cases) more 10 fitness cases of the 16 cases of the last addresses, having a fitness of 310. The chromosome 2301 of this individual and the respective expression tree 2302 are shown in FIG. 23.

In generation 86 an individual was created capable of decoding completely the four addresses of the 6-multiplex (all the 64 fitness cases), having the maximum fitness of 400. The chromosome 2401 of this individual and the respective expression tree 2402 are shown in FIG. 24. This program is, in fact, a universal solution for the 6-multiplexer problem.

It is worth noticing that the problem of the 6-multiplexer was solved by other evolutionary systems, among them genetic programming, but none of them was capable of solving the 6-multiplexer using the set of functions used in this example (AND, OR, NOT). In fact, the present invention is capable of solving the 6-multiplexer with success rates of 100% using the Boolean function if (x,y,z) and the 11-multiplexer with success rates of 57% using the same function.

1. A genetic algorithm for solving problems such as optimization, function finding, planning and logic synthesis, using populations of individuals wherein the linear chromosome (linear entity) of said individuals has a determined length and is composed of one or more genes composed of a head containing symbols that represent functions and arguments and a tail containing symbols representing arguments, being said chromosome expressed as one or more non-linear sub-entities of different sizes and shapes called sub-expression trees, where said sub-expression trees are linked by a chosen function forming an expression tree which is an hierarchical arrangement of said symbols representing functions and arguments of said genetic algorithm comprising iterations of a series of steps, each iteration comprising the following steps:
expression of each said chromosome as said expression tree;
execution of each said expression tree against a set of fitness cases producing a result by performing each said function according to said hierarchical arrangement of functions and arguments;
assigning each said result to respective expression tree, being said result a measure of the fitness of said corresponding individual in solving the problem;
selecting individuals of said population according to said fitness, having individuals with greater fitness higher probability of being selected;
replicating as much said selected individuals as individuals in said population, wherein each said selected individual reproduces new descendants proportionally to said corresponding fitness being said descendants identical copies of corresponding selected individuals;
choosing and executing one or several operators, wherein each said chosen operator belongs to a set of operators comprising mutation, transposition, insertion, gene transposition, one-point recombination and two-point recombination;
if said chosen operator is mutation, said descendant is modified by changing at least one said symbol of said replicated chromosome for another without disrupting the structural and functional organization of said head and said tail of said genes producing a new descendant;
if said chosen operator is transposition, said descendant is modified by intra-chromosomal transposition of transposition elements randomly chosen among said symbols of said head to the start of a randomly chosen gene of said replicated chromosome without disrupting the structural and functional organization of said head and said tail of said chosen gene producing a new descendant;
if said chosen operator is insertion, said descendant is modified by intra-chromosomal insertion of insertion elements randomly chosen among said symbols of said replicated chromosome to said head of a randomly chosen gene without disrupting the structural and functional organization of said head and said tail of said chosen gene producing a new descendant;
if said chosen operator is gene transposition, said descendant is modified by intra-chromosomal transposition of a randomly chosen entire gene to start of said replicated chromosome producing a new descendant;
if said chosen operator is one-point recombination, at least two said replicated chromosomes are randomly chosen and paired to be modified by exchanging the material downstream the recombination point of said chosen replicated chromosomes producing two new descendants;
if said chosen operator is two-point recombination, at least two said replicated chromosomes are randomly chosen and paired to be modified by exchanging an entire gene producing two new descendants;
adding said new descendants to said population.

2. A genetic algorithm as set forth in claim 1, wherein said selection step further comprises a selection scheme that selects, most of the times, individuals according to said fitness as a random factor is incorporated in said selection scheme.

3. A genetic algorithm as set forth in claim 1, further comprising a selection and replication step wherein the individual with said higher fitness is selected and replicated forming a new descendant.

4. A genetic algorithm as set forth in claim 1, wherein an individual of said population having a pre-established value of fitness is the solution to the problem.

5. A genetic algorithm as set forth in claim 1, wherein the initial population of individuals is randomly generated creating chromosomes composed of one or more genes composed of a head containing symbols that represent functions and arguments and a tail containing symbols representing arguments.

6. In a computer system with a population of programs expressed as expression trees of different sizes and shapes, an iterative genetic algorithm comprising iterations of a series of steps, each iteration of said genetic algorithm comprising the steps:
expression of each said program as said expression tree;
execution of each said expression tree to produce a result;
assigning each said result to respective expression tree, being said result a measure of the fitness of said corresponding program in solving the problem;
selecting programs of said population according to said fitness, having programs with greater fitness higher probability of being selected;
replicating as much said selected programs as programs in said population, wherein each said selected program reproduces new programs proportionally to said corresponding fitness being said new programs identical copies of corresponding selected programs;
choosing and executing one or several operators, wherein each said chosen operator belongs to a set of operators comprising mutation, transposition, insertion, gene transposition, one-point recombination and two-point recombination;
if said chosen operator is mutation, said new program is modified by changing at least one said symbol of said replicated program for another without disrupting the structural and functional organization of said head and said tail of said genes producing a new program;
if said chosen operator is transposition, said new program is modified by intra-chromosomal transposition of transposition elements randomly chosen among said symbols of said head to the start of a randomly chosen gene of said replicated program without disrupting the structural and functional organization of said head and said tail of said chosen gene producing a new program;
if said chosen operator is insertion, said new program is modified by intra-chromosomal insertion of insertion elements randomly chosen among said symbols of said replicated program to said head of a randomly chosen gene without disrupting the structural and functional organization of said head and said tail of said chosen gene producing a new program;
if said chosen operator is gene transposition, said new program is modified by intra-chromosomal transposition of a randomly chosen entire gene to start of said replicated program producing a new program;
if said chosen operator is one-point recombination, at least two said replicated programs are randomly chosen and paired to be modified by exchanging the material downstream the recombination point of said chosen replicated programs producing two new programs;
if said chosen operator is two-point recombination, at least two said replicated programs are randomly chosen and paired to be modified by exchanging an entire gene producing two new programs;
adding said new programs to said population.

7. A genetic algorithm as set forth in claim 6, wherein said selection step further comprises a selection scheme that selects, most of the times, programs according to said fitness as a random factor is incorporated in said selection scheme.

8. A genetic algorithm as set forth in claim 6, further comprising a selection and replication step wherein the program with said higher fitness is selected and replicated forming a new program.

9. A genetic algorithm as set forth in claim 6, wherein a program of said population having a pre-established value of fitness is the solution to the problem.

10. A genetic algorithm as set forth in claim 6, wherein the initial population of programs is randomly generated creating programs composed of one or more genes composed of a head containing symbols that represent functions and arguments and a tail containing symbols representing arguments.

11. In a parallel processing computer system with a population of programs expressed as expression trees of different sizes and shapes where more than one program can be executed simultaneously, a set of parallel genetic algorithms, wherein more than one genetic algorithm of said set of genetic algorithms can be executed simultaneously, each said parallel genetic algorithm comprising iterations of a series of steps, each iteration of said parallel genetic algorithm comprising the steps:
expression of each said program as said expression tree;
execution of each said expression tree to produce a result;
assigning each said result to respective expression tree, being said result a measure of the fitness of said corresponding program in solving the problem;
selecting programs of said population according to said fitness, having programs with greater fitness higher probability of being selected;
replicating as much said selected programs as programs in said population, wherein each said selected program reproduces new programs proportionally to said corresponding fitness being said new programs identical copies of corresponding selected programs;
choosing and executing one or several operators, wherein each said chosen operator belongs to a set of operators comprising mutation, transposition, insertion, gene transposition, one-point recombination and two-point recombination;
if said chosen operator is mutation, said new program is modified by changing at least one said symbol of said replicated program for another without disrupting the structural and functional organization of said head and said tail of said genes producing a new program;
if said chosen operator is transposition, said new program is modified by intra-chromosomal transposition of transposition elements randomly chosen among said symbols of said head to the start of a randomly chosen gene of said replicated program without disrupting the structural and functional organization of said head and said tail of said chosen gene producing a new program;
if said chosen operator is insertion, said new program is modified by intra-chromosomal insertion of insertion elements randomly chosen among said symbols of said replicated program to said head of a randomly chosen gene without disrupting the structural and functional organization of said head and said tail of said chosen gene producing a new program;
if said chosen operator is gene transposition, said new program is modified by intra-chromosomal transposition of a randomly chosen entire gene to start of said replicated program producing a new program;
if said chosen operator is one-point recombination, at least two said replicated programs are randomly chosen and paired to be modified by exchanging the material downstream the recombination point of said chosen replicated programs producing two new programs;
if said chosen operator is two-point recombination, at least two said replicated programs are randomly chosen and paired to be modified by exchanging an entire gene producing two new programs;
adding said new programs to said population.

12. A genetic algorithm as set forth in claim 11, wherein said selection step further comprises a selection scheme that selects, most of the times, programs according to said fitness as a random factor is incorporated in said selection scheme.

13. A genetic algorithm as set forth in claim 11, further comprising a selection and replication step wherein the program with said higher fitness is selected and replicated forming a new program.

14. A genetic algorithm as set forth in claim 11, wherein a program of said population having a pre-established value of fitness is the solution to the problem.

15. A genetic algorithm as set forth in claim 11, wherein the initial population of programs is randomly generated creating programs composed of one or more genes composed of a head containing symbols that represent functions and arguments and a tail containing symbols representing arguments.
